SEQUENCE LISTING (9 sequences)

SEQ ID NO: 1 - protein, 28 aa, Homo sapiens
SLRRSSCFGGRMDRIGAQSGLGCNSFRY

SEQ ID NO: 2 - protein, 12 aa, Artificial Sequence
  synthetic polypeptide
RITAREDKQGWA

SEQ ID NO: 3 - protein, 40 aa, Artificial Sequence
  synthetic polypeptide
SLRRSSCFGGRMDRIGAQSGLGCNSFRYRITAREDKQGWA

SEQ ID NO: 4 - DNA, 93 bp, Homo sapiens
agcctgcggagatccagctgcttcgggggcaggatggacaggattggagcccagagcggactgggctgtaacagcttccggtaccgaagataa

SEQ ID NO: 5 - DNA, 123 bp, Artificial Sequence
  synthetic oligonucleotide
agcctgcggagatccagctgcttcgggggcaggatggacaggattggagcccagagcggactgggctgtaacagcttccggtaccggataacagccagggaggacaagcagggctgggcctag

SEQ ID NO: 6 - protein, 39 aa, Artificial Sequence
  synthetic polypeptide
SLRRSSCFGGRMDRIGAQSGLGCNSFRYRIAREDKQGWA

SEQ ID NO: 7 - protein, 40 aa, Artificial Sequence
  synthetic polypeptide
SLRRSSCFGGRMDRIGAQSGLGCNSFRYRITAREDKQGYA

SEQ ID NO: 8 - protein, 41 aa, Artificial Sequence
  synthetic polypeptide
SLRRSSCFGGRMDRIGAQSGLGCNSFRYRITAREDKSQGWA

SEQ ID NO: 9 - protein, 9 aa, Artificial Sequence
  synthetic polypeptide
RITAREDKQ

(19) United States
(12) Patent Application Publication
(10) Pub. No.: US 2014/0066367 A1
(43) Pub. Date: Mar. 6, 2014

(54) SUBCUTANEOUS DELIVERY OF A LONG-ACTING NATRIURETIC PEPTIDE

(71) Applicant: Mayo Foundation for Medical Education and Research, Rochester, MN (US)

(72) Inventors: Horng H. Chen, Rochester, MN (US); John C. Burnett, JR., Rochester, MN (US); Daniel J. McCormick, Rochester, MN (US)

(73) Assignee: MAYO FOUNDATION FOR MEDICAL EDUCATION AND RESEARCH, Rochester, MN (US)

(21) Appl. No.: 13/795,256

(22) Filed: Mar. 12, 2013

Related U.S. Application Data
(60) Provisional application No. 61/695,400, filed on Aug. 31, 2012.

(51) Int. Cl. A61K 38/22; A61K 45/06

(52) U.S. Cl. CPC A61K 38/2242; A61K 45/06
USPC 514/4.8; 514/12.4; 514/6.9; 514/6.5; 530/324

(57) ABSTRACT

This document provides methods and material related to natriuretic polypeptides. For example, substantially pure polypeptides having a natriuretic peptide activity, nucleic acids encoding polypeptides having a natriuretic peptide activity, host cells containing such nucleic acids, and methods for inducing a natriuretic or diuretic activity within a mammal are provided.

### CROSS-REFERENCE TO RELATED APPLICATIONS

This application claims benefit of priority from U.S. Provisional Application Ser. No. 61/695,400, filed on Aug. 31, 2012.

### STATEMENT AS TO FEDERALLY SPONSORED RESEARCH

This invention was made with government support under HL036634 and HL076611, awarded by the National Institutes of Health. The government has certain rights in the invention.

### BACKGROUND

1. Technical Field

This document relates to methods and materials that can be used to treat cardiovascular and metabolic disorders.

2. Background Information

Natriuretic polypeptides are polypeptides that can cause natriuresis (e.g., excretion of an excessively large amount of sodium in the urine). Such polypeptides can be produced by brain, heart, and/or vasculature tissue. Natriuretic polypeptides also may be diuretic.

### SUMMARY

Methods and materials related to natriuretic polypeptides such as atrial natriuretic peptide (ANP), and particularly to M-ANP (an ANP based peptide) are provided herein. For example, this document provides substantially pure M-ANP polypeptides having a natriuretic activity, compositions containing M-ANP with or without other polypeptides or compounds, nucleic acids encoding M-ANP, host cells containing such nucleic acids, and methods for using M-ANP to treat cardiovascular and/or metabolic disorders in a mammal.

This disclosure is based in part on the discovery that subcutaneously administered M-ANP is biologically active (e.g., is diuretic and natriuretic) and lowers blood pressure, and is even more effective than ANP. ANP has been used to regulate blood pressure in mammals, but therapeutic use of ANP has been restricted to intravenous administration due to rapid degradation. The results presented herein provided evidence that M-ANP can be used in chronic delivery strategies, and laid the foundation for studies assessing the therapeutic properties of subcutaneous M-ANP in cardiovascular and metabolic disease.

In one aspect, this disclosure features a method for treating a cardiovascular or metabolic disorder in a mammal. The method can include subcutaneously administering to the mammal a polypeptide comprising an amino acid sequence as set forth in SEQ ID NO:3, or an amino acid sequence as set forth in SEQ ID NO:3 having less than five amino acid additions, subtractions, and substitutions. The polypeptide can comprise an amino acid sequence as set forth in SEQ ID NO:3 having less than four amino acid additions, subtractions, and substitutions. In some embodiments, the polypeptide can consist of the amino acid sequence set forth in SEQ ID NO:3. The cardiovascular disorder can be selected from the group consisting of hypertension, resistant hypertension, and myocardial infarction. The metabolic disorder can be selected from the group consisting of obesity and type II diabetes. The method can include administering the polypeptide via depot polymer, transdermal patch, injection, pump, microparticle, or nanoparticle. The method can further include administering to the mammal insulin and/or an aldosterone inhibitor (e.g., spironolactone or eplerenone). The polypeptide and the insulin and/or aldosterone inhibitor can be administered simultaneously or sequentially. The polypeptide can be coupled to a fatty acid.

In another aspect, this disclosure features a method for treating a cardiovascular or metabolic disorder in a mammal. The method can include subcutaneously administering to the mammal a composition containing a polypeptide that comprises an amino acid sequence as set forth in SEQ ID NO:3, or an amino acid sequence as set forth in SEQ ID NO:3 having less than five amino acid additions, subtractions, and substitutions. The polypeptide can comprise an amino acid sequence as set forth in SEQ ID NO:3 having less than four amino acid additions, subtractions, and substitutions. The polypeptide can consist of the amino acid sequence set forth in SEQ ID NO:3. The cardiovascular disorder can be selected from the group consisting of hypertension, resistant hypertension, and myocardial infarction. The metabolic disorder can be selected from the group consisting of obesity and type II diabetes. The method can include administering the composition via depot polymer, transdermal patch, injection, pump, microparticle, or nanoparticle. The method can further include administering to the mammal insulin and/or an aldosterone inhibitor (e.g., spironolactone or eplerenone). In such embodiments, the composition can contain the polypeptide and the insulin and/or aldosterone inhibitor, or the composition and the insulin or aldosterone inhibitor can be administered separately. The polypeptide can be coupled to a fatty acid.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a representation of the structures of ANP (left) and M-ANP (right), showing the amino acid sequences of both peptides. The amino acid sequence of M-ANP includes the 28 amino acids of native ANP (white) and a 12 amino acid addition (gray) to the C-terminus.

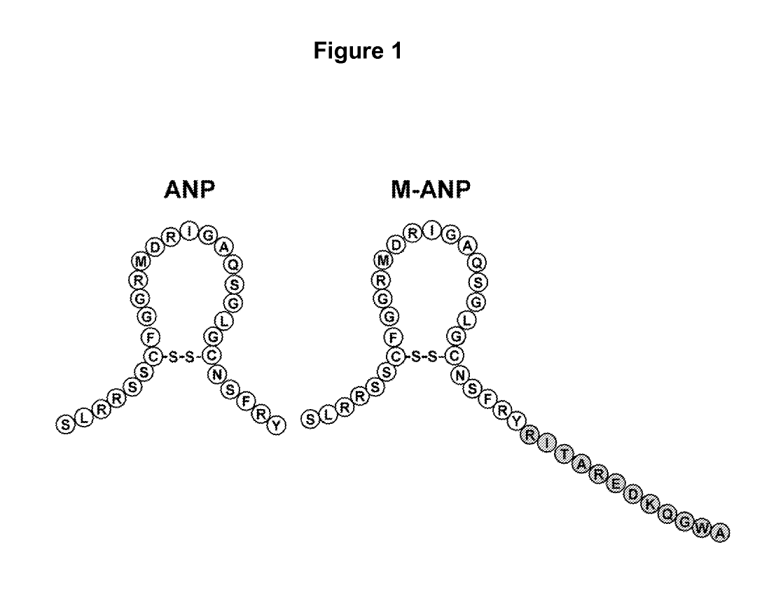

FIG. 2A is a graph plotting in vitro 3′,5′-cyclic guanosine monophosphate (cGMP) generation in human embryonic kidney 293 cells stably transfected with guanylyl cyclase A in response to ANP (white bar) and M-ANP (hatched bar) compared with vehicle (no treatment; black bar). Values are mean±SEM. *p<0.01 compared to vehicle (t-test).

FIG. 2B is a graph plotting in vitro 3′,5′-cGMP generation in human embryonic kidney 293 cells stably transfected with guanylyl cyclase B in response to ANP (white bar) and M-ANP (hatched bar) compared with vehicle (no treatment; black bar). Values are mean±SEM. *p<0.01 compared to vehicle (t-test).

FIG. 3A is a graph plotting plasma ANP immunoreactivity following equimolar subcutaneous administration of M-ANP, ANP, and vehicle to normal conscious canines *p<0.05 vs. time 0 minutes, 1-way ANOVA with Bonferroni multiple comparison test. †p<0.05 for M-ANP or ANP vs. vehicle and ‡p<0.05 for M-ANP vs. ANP at a specific time point, 2-way ANOVA with Bonferroni posttest. P<0.001 between M-ANP and ANP groups (2-way ANOVA).

FIG. 3B is a graph plotting plasma cGMP immunoreactivity following equimolar subcutaneous administration of M-ANP, ANP, and vehicle to normal conscious canines *p<0.05 vs. time 0 minutes, 1-way ANOVA with Bonferroni multiple comparison test. †p<0.05 for M-ANP or ANP vs. vehicle and ‡p<0.05 for M-ANP vs. ANP at a specific time point, 2-way ANOVA with Bonferroni posttest. P<0.001 between M-ANP and ANP groups (2-way ANOVA).

FIG. 4 is a graph plotting in vivo cGMP activation for given levels of plasma ANP immunoreactivity following subcutaneous M-ANP and ANP administration. Linear regression lines are shown (r² was 0.59 for M-ANP and 0.21 for ANP). The slope was significantly greater for M-ANP than for ANP (p<0.01; analysis of co-variance).

FIG. 5A is a graph plotting mean arterial pressure (MAP) following equimolar subcutaneous administration of M-ANP, ANP, and vehicle to normal conscious canines *p<0.05 vs. time 0 minutes, 1-way ANOVA with Bonferroni multiple comparison test. †p<0.05 for M-ANP or ANP vs. vehicle and ‡p<0.05 for M-ANP vs. ANP at a specific time point, 2-way ANOVA with Bonferroni posttest. P<0.001 between M-ANP and ANP groups (2-way ANOVA).

FIG. 5B is a graph plotting heart rate following equimolar subcutaneous administration of M-ANP, ANP, and vehicle to normal conscious canines. P>0.05 between M-ANP and ANP groups (2-way ANOVA).

FIG. 6 is a representation of the structure of M-ANP with palmitic acid attached to the N-terminal serine.

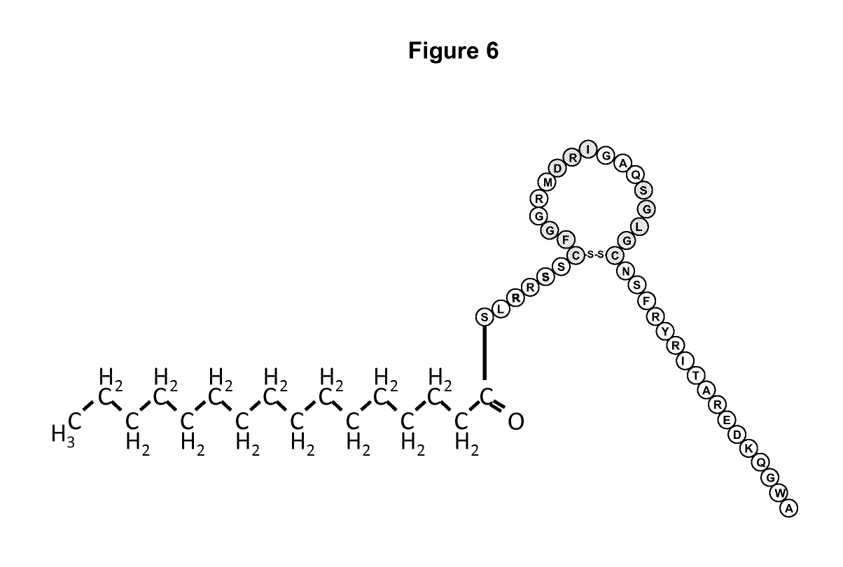

### DETAILED DESCRIPTION

This disclosure provides methods and materials related to natriuretic polypeptides, and particularly to M-ANP. For example, this document provides substantially pure polypeptides having a natriuretic polypeptide activity, nucleic acid molecules encoding polypeptides having a natriuretic polypeptide activity, and host cells containing isolated nucleic acid molecules that encode polypeptides having a natriuretic polypeptide activity. In addition, this document provides methods and materials for treating a cardiovascular or metabolic disorder in a mammal.

### Polypeptides

The term “substantially pure” as used herein with reference to a polypeptide means the polypeptide is substantially free of other polypeptides, lipids, carbohydrates, and nucleic acid with which it is naturally associated. A substantially pure polypeptide can be any polypeptide that is removed from its natural environment and is at least 60 percent pure. A substantially pure polypeptide can be at least about 65, 70, 75, 80, 85, 90, 95, or 99 percent pure. Typically, a substantially pure polypeptide will yield a single major band on a non-reducing polyacrylamide gel. A substantially pure polypeptide can be a chemically synthesized polypeptide.

Any method can be used to obtain a substantially pure polypeptide. For example, common polypeptide purification techniques such as affinity chromotography and HPLC as well as polypeptide synthesis techniques can be used. In addition, any material can be used as a source to obtain a substantially pure polypeptide. For example, tissue from wild-type or transgenic animals can be used as a source material. In addition, tissue culture cells engineered to over-express a particular polypeptide can be used to obtain substantially pure polypeptide. Further, a polypeptide can be engineered to contain an amino acid sequence that allows the polypeptide to be captured onto an affinity matrix. For example, a tag such as c-myc, hemagglutinin, polyhistidine, or Flag™ tag (Kodak) can be used to aid polypeptide purification. Such tags can be inserted anywhere within the polypeptide including at either the carboxyl or amino termini, or in between. Other fusions that can be used include enzymes that aid in the detection of the polypeptide, such as alkaline phosphatase.

A substantially pure polypeptide provided herein can contain one or more sequences present in a polypeptide having natriuretic polypeptide activity. Examples of polypeptides having a natriuretic polypeptide activity (e.g., an ANP activity) include, without limitation, brain natriuretic peptide (BNP), C-type natriuretic peptide (CNP), urodilatin, snake natriuretic peptide (DNP), and ANP polypeptides. The polypeptides having a natriuretic polypeptide activity can have a non-naturally occurring sequence or can have a sequence present in any species (e.g., human, horse, pig, goat, cow, dog, cat, rat, or mouse). For example, a polypeptide having a natriuretic polypeptide activity can be a human ANP polypeptide having one or more amino acid changes.

A polypeptide having natriuretic activity also can be an M-ANP peptide. M-ANP is an ANP-based peptide having an amino acid sequence that includes the 28 amino acid mature human ANP sequence (SLRRSSCFGGRMDRIGAQSGLGCNSFRY; SEQ ID NO:1) with an additional 12 amino acid carboxy terminus (RITAREDKQGWA; SEQ ID NO:2). The full length sequence of M-ANP is SLRRSSCFGGRMDRIGAQSGLGCNSF RYRITAREDKQGWA (SEQ ID NO:3). The nucleic acid sequence 5′-agcctgcggagatcc agctgcttcgggggcaggatggacaggattggagcccagagcggactgggctgtaacagcttccggtaccgaagataa-3′ (SEQ ID NO:4) encodes human ANP, and the nucleic acid sequence 5′-agcctgcggagatcca gctgcttcgggggcaggatggacaggattggagcccagagcggactgggctgtaacagcttccggtaccggataacagccag ggaggacaagcagggctgggcctag-3′ (SEQ ID NO:5) encodes M-ANP.

As described in U.S. Pat. Nos. 7,803,901 and 8,063,191 (which are incorporated herein by reference in their entirety), studies comparing M-ANP to ANP in normal dogs showed that M-ANP had greater diuretic and natriuretic effects than ANP. Low dose M-ANP also had a greater effect than low dose ANP on renal blood flow. No change in glomerular filtration rate (GFR) or MAP was observed during infusion of a low dose of M-ANP or ANP, whereas infusion of a high dose of M-ANP increased GFR and decreased MAP as compared to the effects of a high dose of ANP.

The polypeptides provided herein can contain the entire amino acid sequence set forth in SEQ ID NO:3. In some cases, a polypeptide can contain the amino acid sequence set forth in SEQ ID NO:3 except that the amino acid sequence contains one or between one and ten (e.g., ten, between one and nine, between two and nine, between one and eight, between two and eight, between one and seven, between one and six, between one and five, between one and four, between one and three, two, or one) amino acid additions, subtractions, and substitutions. For example, a polypeptide can contain the amino acid sequence set forth in SEQ ID NO:3 with one, two, three, four, five, six, seven, eight, nine, or ten single amino acid residue additions, subtractions, or substitutions. Examples of such a polypeptide include, without limitation, a polypeptide having the amino acid sequence set forth in SEQ ID NO:3 where the threonine is deleted (SEQ ID NO:6), the tryptophan is replaced with a tyrosine (SEQ ID NO:7), a serine is added between the lysine and the glutamine (SEQ ID NO:8), or any combination thereof. Another example can be a polypeptide containing a contiguous amino acid sequence that is identical to the first nine amino acid residues set forth in SEQ ID NO:2 and lacking last three residues (i.e., the glycine, tryptophan, and alanine residues; SEQ ID NO:9).

Any amino acid residue set forth in SEQ ID NO:3 can be subtracted, and any amino acid residue (e.g., any of the 20 conventional amino acid residues or any other type of amino acid such as ornithine or citrulline) can be added to the sequence set forth in SEQ ID NO:3. In some cases, a polypeptide provided herein can contain chemical structures such as ε-aminohexanoic acid; hydroxylated amino acids such as 3-hydroxyproline, 4-hydroxyproline, (5R)-5-hydroxy-L-lysine, allo-hydroxylysine, and 5-hydroxy-L-norvaline; or glycosylated amino acids such as amino acids containing monosaccharides (e.g., D-glucose, D-galactose, D-mannose, D-glucosamine, and D-galactosamine) or combinations of monosaccharides.

Polypeptides having one or more amino acid substitutions relative to a native polypeptide can be prepared and modified as described herein. Amino acid substitutions can be conservative amino acid substitutions. Conservative amino acid substitutions include, for example, aspartic acid/glutamic acid as acidic amino acids; lysine/arginine/histidine as basic amino acids; leucine/isoleucine, methionine/valine, alanine/valine as hydrophobic amino acids; serine/glycine/alanine/threonine as hydrophilic amino acids. In some cases, amino acid substitutions can be substitutions that do not differ significantly in their effect on maintaining (a) the structure of the peptide backbone in the area of the substitution, (b) the charge or hydrophobicity of the molecule at the target site, or (c) the bulk of the side chain. Thus, conservative amino acid substitutions also can include groupings based on side chain properties. For example, a group of amino acids having aliphatic side chains is glycine, alanine, valine, leucine, and isoleucine; a group of amino acids having hydrophobic side chains is norleucine, methionine, alanine, valine, leucine, and isoleucine; a group of amino acids having neutral hydrophilic side chains is cysteine, serine, and threonine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is asparagine, glutamine, lysine, arginine, and histidine; a group of amino acids having acidic side chains is aspartic acid and glutamic acid; a group of amino acids having side chains with residues that influence chain orientation is glycine, and proline; a group of amino acids having aromatic side chains is tryptophan, tyrosine, and phenylalanine; and a group of amino acids having sulfur-containing side chains is cysteine and methionine. It is reasonable to expect that replacement of a leucine with an isoleucine or valine, an aspartate with a glutamate, a threonine with a serine, or a similar replacement of an amino acid with a structurally related amino acid will not have a major effect on the properties of the resulting polypeptide. Whether an amino acid change results in a functional polypeptide can readily be determined by assaying the activity of the polypeptide.

In some cases, non-conservative substitutions can be used. A non-conservative substitution can include exchanging a member of one of the classes described herein for another.

In some cases, a polypeptide provided herein can be pegylated, acetylated, or both. In some cases, a polypeptide provided herein can be covalently attached to oligomers, such as short, amphiphilic oligomers that enable administration or improve the pharmacokinetic or pharmacodynamic profile of the conjugated polypeptide. The oligomers can comprise water soluble polyethylene glycol (PEG) and lipid soluble alkyls (short, medium, or long chain fatty acid polymers, such as, without limitation, palmitic acid, myristic acid, lauric acid, capric acid, or steric acid). See, for example, PCT Publication No. WO 2004/047871. The fatty acid molecule can be attached to the free amino terminus or to any lysine side chain (an epsilon amino group), and a lysine residue for this attachment can be placed at either the C-terminal or N-terminal end of the peptide

In some cases, a polypeptide provided herein can be fused to the Fc domain of an immunoglobulin molecule (e.g., an IgG1 molecule) such that active transport of the fusion polypeptide across epithelial cell barriers occurs via the Fc receptor. In some cases, a polypeptide can be a cyclic polypeptide. A cyclic polypeptide provided herein can be obtained by bonding cysteine residues, however, the replacement of a sulfhydryl group on the cysteine residue with an alternative group also is envisioned, for example, —CH₂—CH₂—. For example, to replace sulfhydryl groups with a —CH₂— group, the cysteine residues can be replaced by the analogous alpha-aminobutyric acid. These cyclic analog peptides can be formed, for example, in accordance with the methodology of Lebl and Hruby (Tetrahedron Lett., 25:2067-2068, 1984), or by employing the procedure disclosed in U.S. Pat. No. 4,161,521.

A substantially pure polypeptide provided herein can have a natriuretic peptide activity. For example, a polypeptide provided herein can have natriuretic or diuretic activity. Any method can be use to determine whether or not a particular polypeptide has a natriuretic peptide activity. For example, a mammal (e.g., dog or human) exposed to a particular polypeptide can be analyzed to determine the polypeptide's ability to induce natriuretic or diuretic activity.

### Nucleic Acids, Vectors, and Host Cells

This document also provides nucleic acid molecules having a sequence that encodes any of the polypeptides provided herein. For example, this document provides nucleic acid molecules that encode a natriuretic peptide containing the amino acid sequence set forth in SEQ ID NO:3. In some cases, a nucleic acid molecule provided herein can encode a polypeptide that contains the amino acid sequence set forth in SEQ ID NO:3, except that the amino acid sequence contains one or between one and ten (e.g., ten, between one and nine, between two and nine, between one and eight, between two and eight, between one and seven, between one and six, between one and five, between one and four, between one and three, two, or one) amino acid additions, subtractions, and substitutions as described herein.

The term “nucleic acid” as used herein encompasses both RNA and DNA, including cDNA, genomic DNA, and synthetic (e.g., chemically synthesized) DNA. The nucleic acid can be double-stranded or single-stranded. Where single-stranded, the nucleic acid can be the sense strand or the antisense strand. In addition, nucleic acid can be circular or linear.

The term “isolated” as used herein with reference to nucleic acid refers to a naturally-occurring nucleic acid that is not immediately contiguous with both of the sequences with which it is immediately contiguous (one on the 5′ end and one on the 3′ end) in the naturally-occurring genome of the organism from which it is derived. For example, an isolated nucleic acid can be, without limitation, a recombinant DNA molecule of any length, provided one of the nucleic acid sequences normally found immediately flanking that recombinant DNA molecule in a naturally-occurring genome is removed or absent. Thus, an isolated nucleic acid includes, without limitation, a recombinant DNA that exists as a separate molecule (e.g., a cDNA or a genomic DNA fragment produced by PCR or restriction endonuclease treatment) independent of other sequences as well as recombinant DNA that is incorporated into a vector, an autonomously replicating plasmid, a virus (e.g., a retrovirus, adenovirus, or herpes virus), or into the genomic DNA of a prokaryote or eukaryote. In addition, an isolated nucleic acid can include a recombinant DNA molecule that is part of a hybrid or fusion nucleic acid sequence.

The term “isolated” as used herein with reference to nucleic acid also includes any non-naturally-occurring nucleic acid since non-naturally-occurring nucleic acid sequences are not found in nature and do not have immediately contiguous sequences in a naturally-occurring genome. For example, non-naturally-occurring nucleic acid such as an engineered nucleic acid is considered to be isolated nucleic acid. Engineered nucleic acid can be made using common molecular cloning or chemical nucleic acid synthesis techniques. Isolated non-naturally-occurring nucleic acid can be independent of other sequences, or incorporated into a vector, an autonomously replicating plasmid, a virus (e.g., a retrovirus, adenovirus, or herpes virus), or the genomic DNA of a prokaryote or eukaryote. In addition, a non-naturally-occurring nucleic acid can include a nucleic acid molecule that is part of a hybrid or fusion nucleic acid sequence.

It will be apparent to those of skill in the art that a nucleic acid existing among hundreds to millions of other nucleic acid molecules within, for example, cDNA or genomic libraries, or gel slices containing a genomic DNA restriction digest is not to be considered an isolated nucleic acid.

In some cases, an isolated nucleic acid molecule provided herein can be at least about 12 bases in length (e.g., at least about 13, 14, 15, 16, 17, 18, 19, 20, 25, 30, 40, 50, 60, 100, 250, 500, 750, 1000, 1500, 2000, 3000, 4000, or 5000 bases in length) and hybridize, under hybridization conditions, to the sense or antisense strand of a nucleic acid having a sequence that encodes the sequence set forth in SEQ ID NO:3. The hybridization conditions can be moderately or highly stringent hybridization conditions. In some cases, such nucleic acid molecules can be molecules that do not hybridize to the sense or antisense strand of a nucleic acid that consists only of the coding sequence of a natriuretic peptide such as human ANP, human BNP, or human CNP.

For the purpose of this document, moderately stringent hybridization conditions mean the hybridization is performed at about 42° C. in a hybridization solution containing 25 mM KPO₄ (pH 7.4), 5×SSC, 5×Denhart's solution, 50 μg/mL denatured, sonicated salmon sperm DNA, 50% formamide, 10% Dextran sulfate, and 1-15 ng/mL probe (about 5×10⁷ cpm/m), while the washes are performed at about 50° C. with a wash solution containing 2×SSC and 0.1% sodium dodecyl sulfate.

Highly stringent hybridization conditions mean the hybridization is performed at about 42° C. in a hybridization solution containing 25 mM KPO₄ (pH 7.4), 5×SSC, 5×Denhart's solution, 50 μg/mL denatured, sonicated salmon sperm DNA, 50% formamide, 10% Dextran sulfate, and 1-15 ng/mL probe (about 5×10⁷ cpm/m), while the washes are performed at about 65° C. with a wash solution containing 0.2×SSC and 0.1% sodium dodecyl sulfate.

An isolated nucleic acid molecule provided herein can be obtained using any method including, without limitation, common molecular cloning and chemical nucleic acid synthesis techniques. For example, PCR can be used to obtain an isolated nucleic acid molecule containing a nucleic acid sequence sharing similarity to sequences that encode the amino acid sequence set forth in SEQ ID NO:3. PCR refers to a procedure or technique in which target nucleic acid is amplified in a manner similar to that described in U.S. Pat. No. 4,683,195, and subsequent modifications of the procedure described therein. Generally, sequence information from the ends of the region of interest or beyond are used to design oligonucleotide primers that are identical or similar in sequence to opposite strands of a potential template to be amplified. Using PCR, a nucleic acid sequence can be amplified from RNA or DNA. For example, a nucleic acid sequence can be isolated by PCR amplification from total cellular RNA, total genomic DNA, and cDNA as well as from bacteriophage sequences, plasmid sequences, viral sequences, and the like. When using RNA as a source of template, reverse transcriptase can be used to synthesize complementary DNA strands.

An isolated nucleic acid molecule provided herein can be obtained by mutagenesis. For example, an isolated nucleic acid containing a sequence that encodes the amino acid sequence set forth in SEQ ID NO:3 can be mutated using common molecular cloning techniques (e.g., site-directed mutagenesis). Possible mutations include, without limitation, additions, subtractions, and substitutions, as well as combinations of additions, subtractions, and substitutions. Such methods can be used to obtain a nucleic acid encoding a polypeptide containing the amino acid sequence set forth in SEQ ID NO:3, but with one to ten amino acid substitutions, additions, or deletions.

This document also provides host cells containing at least one of the isolated nucleic acid molecules provided herein. For example, host cells can contain an exogenous nucleic acid molecule that encodes a polypeptide provided herein (e.g., a polypeptide containing an amino acid sequence as set forth in SEQ ID NO:3). Such host cells can express the encoded polypeptide, but it is noted that cells containing an isolated nucleic acid molecule provided herein are not required to express a polypeptide. Host cells can be prokaryotic or eukaryotic cells. In addition, the isolated nucleic acid molecule can be integrated into the genome of the cell or maintained in an episomal state. Thus, host cells can be stably or transiently transfected with a construct containing an isolated nucleic acid molecule provided herein.

Any method can be used to introduce an isolated nucleic acid molecule into a cell in vivo or in vitro. For example, calcium phosphate precipitation, electroporation, heat shock, lipofection, microinjection, and viral-mediated nucleic acid transfer are methods that can be used to introduce an isolated nucleic acid molecule into a cell. In addition, naked DNA can be delivered directly to cells in vivo as described elsewhere (e.g., U.S. Pat. Nos. 5,580,859 and 5,589,466, and continuations thereof). Further, isolated nucleic acid molecules can be introduced into cells by generating transgenic animals.

Any method can be used to identify cells containing an isolated nucleic acid molecule provided herein. Such methods include, without limitation, PCR and nucleic acid hybridization techniques such as Northern and Southern analyses. In some cases, immunohistochemistry and biochemical techniques can be used to determine if a cell contains a particular isolated nucleic acid molecule by detecting the expression of a polypeptide encoded by that nucleic acid molecule.

### Compositions

Compositions containing natriuretic polypeptides (e.g., M-ANP) also are provided herein. The polypeptides can be admixed, encapsulated, conjugated or otherwise associated with other molecules, molecular structures, or mixtures of compounds such as, for example, liposomes, PEG, receptor targeted molecules, polymeric carriers, or other formulations, for assisting in uptake, distribution and/or absorption. The compositions also can contain ingredients such as those described in U.S. Pat. No. 6,818,619. Such additional ingredients can be polypeptides or non-polypeptides (e.g., buffers). In addition, the polypeptide(s) within a composition provided herein can be in any form, including those described in U.S. Pat. No. 6,818,619.

In some embodiments, a composition also can include insulin and/or an aldosterone inhibitor. Examples of such inhibitors include, without limitation, spironolactone (brand name ALDACTONE) and eplerenone (brand name INSPRA®, Pfizer).

A natriuretic polypeptide (e.g., an M-ANP polypeptide) can be formulated for subcutaneous delivery via depot polymers, drug patch, injection, pump, or microparticle/nano particle. Examples of such delivery methods are in the art.

By way of example and not limitation, PCT Publication No. WO 2008/061355 discloses materials and methods for formulating a polypeptide for delivery in a hydrogel tube. The polypeptide can be mixed with one or more excipients that are pharmaceutically acceptable and are compatible with the polypeptide in amounts suitable for use in the methods described herein. For example, a polypeptide can be combined with one or more excipients such as, without limitation, microcrystalline cellulose, colloidal silicon dioxide, lactose, starch, sorbitol, cyclodextrin, and combinations thereof. The excipient can be a solid, semi-solid, or liquid material that acts as a vehicle, carrier, or medium for the polypeptide. In some embodiments, the polypeptide can be compressed, compacted, or extruded with one or more excipients prior to inserting it into a hydrogel tube. Such formulations can result in a pharmaceutical composition with desirable release properties, improved stability, and/or other desirable properties.

Pharmaceutical compositions also can include auxiliary agents or excipients, such as glidants, dissolution agents, surfactants, diluents, binders, disintegrants, and/or lubricants. For example, dissolution agents can increase the dissolution rate of the polypeptide from the dosage formulation, and can include, for example, organic acids and/or salts of organic acids (e.g., sodium citrate with citric acid). Other examples of excipients useful in such formulations include synthetic, semi-synthetic, modified, and natural polymers (e.g., lactose, dextrose, sucrose, trehalose, sorbitol, mannitol, starches, gum acacia, calcium silicate, microcrystalline cellulose, polyvinylpyrrolidone, cellulose, water, syrup, PEG, cyclodextrin, alkoxy-modified cyclodextrins, hydroxyethylcellulose, hydroxypropylcellulose, microcrystalline cellulose, albumin, dextran, malitol, xylitol, kaolin, and methyl cellulose). The polypeptide also can be mixed with a lubricating agent (e.g., talc, magnesium stearate, stearic acid, or mineral oil, calcium stearate, hydrogenated vegetable oils, sodium benzoate, sodium chloride, leucine carbowax, magnesium lauryl sulfate, or glyceryl monostearate), a wetting agent, an emulsifying and suspending agent, or a preserving agent (e.g., methyl or propyl hydroxybenzoate).

Other agents that can be added to a pharmaceutical composition can alter the pH of the microenvironment on dissolution and establishment of a therapeutically effective plasma concentration profile of the polypeptide. Such agents include salts of inorganic acids and magnesium hydroxide. Other agents that can be used include surfactants and other solubilizing materials.

Useful diluents include, for example, pharmaceutically acceptable inert fillers such as microcrystalline cellulose, lactose, sucrose, fructose, glucose dextrose, or other sugars, dibasic calcium phosphate, calcium sulfate, cellulose, ethylcellulose, cellulose derivatives, kaolin, mannitol, lactitol, maltitol, xylitol, sorbitol, or other sugar alcohols, dry starch, saccharides, dextrin, maltodextrin or other polysaccharides, inositol or combinations thereof. Water-soluble diluents can be particularly useful.

Glidants can be used to improve the flow and compressibility of composition ingredients during processing. Useful glidants include, for example, colloidal silicon dioxide (also referred to as colloidal silica, fumed silica, light anhydrous silicic acid, silicic anhydride, and silicon dioxide fumed).

Surfactants that are suitable for use in the pharmaceutical compositions provided herein include, without limitation, sodium lauryl sulphate, polyethylene stearates, polyethylene sorbitan fatty acid esters, polyoxyethylene castor oil derivatives, polyoxyethylene alkyl ethers, benzyl benzoate, cetrimide, cetyl alcohol, docusate sodium, glyceryl monooleate, glyceryl monostearate, glyceryl palmitostearate, lecithin, medium chain triglycerides, monoethanolamine, oleic acid, poloxarners, polyvinyl alcohol and sorbitan fatty acid esters.

Suitable disintegrants include, for example, starches, sodium starch glycolate, crospovidone, croscarmellose, microcrystalline cellulose, low substituted hydroxypropyl cellulose, pectins, potassium methacrylate-divinylbenzene copolymer, polyvinyl alcohol), thylamide, sodium bicarbonate, sodium carbonate, starch derivatives, dextrin, beta cyclodextrin, dextrin derivatives, magnesium oxide, clays, bentonite, and combinations thereof.

In some embodiments, an M-ANP polypeptide can be incorporated into a hydrogel delivery system. For example, a polypeptide can be formulated for subcutaneous delivery to a patient via a xerogel-hydrogel system that can release the polypeptide in a continuous sustained manner over an extended period of time. See, for example, U.S. Pat. No. 5,226,325, and PCT Publication No. WO 2004/071736.

Liquid polymerizable materials useful in the preparation of hydrogel tubes include a wide variety of polymerizable hydrophilic, and ethylenically unsaturated compounds. See, for example, the compounds listed in PCT Publication No. WO 2008/061355. Mixtures of such hydrophilic monomers typically are used in the polymerization reaction. The type and proportion of monomers are selected to yield a polymer (e.g., a crosslinked homogeneous polymer) that on hydration possesses the desired characteristics (e.g., equilibrium water content (EWC) value and/or pore size) for the contemplated application or use.

In some cases, the polymerization of hydrophilic monomeric mixtures can result in homogeneous hydrophilic copolymers which dissolve, to a varying extent, in an aqueous medium. In such cases, a small amount (e.g., up to about 3 percent) of a copolymerizable polyethylenically unsaturated crosslinking agent can be included in the monomeric mixture to obtain homogeneous crosslinked copolymers that are water-insoluble as well as water-swellable. A slightly crosslinked homopolymer of (hydroxyethyl)methacrylate (HEMA) has an EWC value of about 38%. Crosslinked copolymers of HEMA and N-(2-hydroxypropyl) methacrylamide (HPMA) have EWC values below 38%, while crosslinked copolymers of HEMA and acrylamide exhibit EWC values above 38 w/v %. Therefore, depending on the useful or effective elution rate of the polypeptide, copolymer hydrogels can be customized to elute the polypeptide at the desired rate. Typically, copolymers contain about 15 to about 70 weight % of HEMA units and from about 85 to 30 weight % of a second ethylenic monomer, and thus possess EWC values in the range of from about 20% to about 75%. In some embodiments, a mixture of copolymers can further contain a small amount of a polyethylenically unsaturated crosslinking agent [e.g., ethyleneglycol dimethacrylate (“EDMA”) or trimethylolpropane trimethacrylate (“TMPTMA”)].

In some embodiments, a pharmaceutical composition for controlled release delivery of an M-ANP polypeptide a subject can include (a) a complex of the polypeptide (where the polypeptide has at least one basic functional group) and a polyanion derived from hexahydroxycyclohexane (where the polyanion has at least two negatively charged functional groups); and (b) a pharmaceutically acceptable carrier containing a biodegradable, water-insoluble polymer. Such compositions are described in, for example, PCT Publication No. WO 2006/017852, and can be prepared in the form of solutions, suspensions, dispersions, emulsions, drops, aerosols, creams, semisolids, pastes, capsules, tablets, solid implants, or microparticles, for example. The term “controlled release delivery,” as used herein, refers to continual delivery of a pharmaceutical agent in vivo over a period of time (e.g., several days to weeks or months) following administration. Sustained controlled release delivery of an M-ANP polypeptide can be demonstrated by, for example, continued therapeutic effects of the polypeptide over time (e.g., continued reductions in symptoms over time). Sustained delivery of the polypeptide also can be demonstrated by detecting the presence of the polypeptide in vivo over time. The compositions can provide a low initial burst delivery, followed by stable, controlled release of the polypeptide in vivo for prolonged periods of time (e.g., from days to months).

In such embodiments, a physically and chemically stable complex can form upon appropriate combining of an M-ANP polypeptide and a polyanion. The complex can take the form of a precipitate that is produced upon combining an aqueous preparation of the polypeptide and the polyanion. Optionally, one or more pharmaceutically acceptable excipients can be incorporated into the complex. Such excipients can function as stabilizers for the polypeptide and/or the complex. Non-limiting examples of suitable excipients include sodium bisulfite, p-aminobenzoic acid, thiourea, glycine, methionine, mannitol, sucrose, and PEG.

A stable complex between an M-ANP polypeptide and a polyanion can be incorporated into a pharmaceutically acceptable carrier containing a biodegradable water-insoluble polymer, optionally with one or more excipients. The term “biodegradable water-insoluble polymer” refers to biocompatible and/or biodegradable synthetic and natural polymers that can be used in vivo. The term also is meant to include polymers that are insoluble or become insoluble in water or biological fluid at 37° C. The polymers can be purified (e.g., to remove monomers and oligomers) using techniques known in the art. See, e.g., U.S. Pat. No. 4,728,721. Examples of useful polymers include, without limitation, polylactides, polyglycolides, poly(lactide-co-glycolide)s, polycaprolactones, polydioxanones, polycarbonates, polyhydroxybutyrates, polyalkylene oxalates, polyanhydrides, polyamides, polyesteramides, polyurethanes, polyacetals, polyorthocarbonates, polyphosphazenes, polyhydroxyvalerates, polyalkylene succinates, and polyorthoesters, and copolymers, block copolymers, branched copolymers, terpolymers, and combinations thereof.

Biodegradable water-insoluble polymers also can include end capped, end uncapped, or mixtures of end capped and end uncapped polymers. An end capped polymer generally is defined as having capped carboxyl end groups, while an uncapped polymer has free carboxyl end groups.

Factors to consider when determining suitable molecular weights for the polymer can include desired polymer degradation rate, mechanical strength, and rate of dissolution of polymer in solvent. Useful molecular weights for polymers can be from about 2,000 Daltons to about 150,000 Daltons, for example, with a polydispersity of from 1.1 to 2.8, depending upon which polymer is selected for use.

The pharmaceutically acceptable carrier can be a carrier with environment responsive properties (e.g., thermosensitive, pH sensitive, or electrical sensitive), in the form of an injectable solution or suspension, particle, film, pellet, cylinder, disc, microcapsule, microsphere, nanosphere, microparticle, wafer, micelle, liposome, or any other polymeric configuration useful for drug delivery.

Methods of forming various pharmaceutically acceptable polymer carriers include those that are known in the art. See, for example, U.S. Pat. Nos. 6,410,044; 5,698,213; 6,312,679; 5,410,016; 5,529,914; 5,501,863; 4,938,763; 5,278,201; and 5,278,202; and PCT Publication No. WO 93/16687.

Compositions can be produced when a polypeptide/polyanion complex is dispersed in a polymeric matrix to form a solid implant, which can be injected or implanted into a subject. Such implants can be prepared using conventional polymer melt-processing techniques, such as extrusion, compression molding, and injection molding, for example. Preparations of such implants can be carried out under aseptic conditions, or alternatively by terminal sterilization by irradiation (e.g., using gamma irradiation or electron beam sterilization).

In some embodiments, compositions in the form of microspheres can be produced by encapsulating a polypeptide/polyanion complex in a polymeric carrier, using various biocompatible and/or biodegradable polymers having properties that are suitable for delivery to different biological environments or for effecting specific functions. The rate of dissolution and, therefore, delivery of polypeptide is determined by factors such as the encapsulation technique, polymer composition, polymer crosslinking, polymer thickness, polymer solubility, and size and solubility of polypeptide/polyanion complex.

To prepare such microspheres, a polypeptide/polyanion complex to be encapsulated can be suspended in a polymer solution in an organic solvent, such that the polymer solution completely coats the polypeptide/polyanion complex. The suspension then can be subjected to a microencapsulation technique such as spray drying, spray congealing, emulsion, or solvent evaporation emulsion. For example, the suspended complexes or microparticles along with the polymer in an organic solvent can be transferred to a larger volume of an aqueous solution containing an emulsifier, such that the organic solvent evaporates or diffuses away from the polymer and the solidified polymer encapsulates the polypeptide/polyanion complex.

Emulsifiers useful to prepare encapsulated polypeptide/polyanion complexes include poloxamers and polyvinyl alcohol, for example. Organic solvents useful in such methods include acetic acid, acetone, methylene chloride, ethyl acetate, chloroform, and other non-toxic solvents that will depend on the properties of the polymer. Solvents typically are chosen that solubilize the polymer and are ultimately non-toxic.

In some embodiments, a polypeptide can be formulated in a depot, which can provide constantly high exposure levels and may reach high exposure levels rapidly (with a short or no lag phase). See, e.g., U.S. Publication No. 2010/0266704. Depot formulations can include an M-ANP polypeptide or a pharmaceutically-acceptable salt thereof (e.g., an acid addition salt with an inorganic acid, polymeric acid, or organic acid). Acid addition salts can exist as mono- or divalent salts, depending on whether one or two acid equivalents are added.

As described in U.S. Publication No. 2010/0266704, depot formulations can contain two different linear poly(lactic-co-glycolic acid) (PLGA) polymers having a molar ratio of lactide:glycolide comonomer (L:G) from 85:15 to 65:35, where at least one of the polymers has a low inherent viscosity. Such formulations can provide sustained high plasma levels of the polypeptide for extended periods of time. Examples of suitable polymers include the linear poly(D,L-lactide) and poly(D,L-lactide-co-glycolide) polymers sold under the trade names RESOMER®, LACTEL®, and MEDISORB® by Boehringer Ingelheim Pharma GmBH & Co. KG (Ingelheim, Germany), Absorbable Polymers International (Pelham, Ala.), and Alkermes, Inc. (Cambridge, Mass.), respectively.

High exposure depot formulations for subcutaneous administration can show immediate or at least very rapid action, such that therapeutic plasma concentrations are achieved in a short time (e.g., one, two, three, four, five, six, or seven days after subcutaneous injection), and can show constantly high exposure levels over about one month or longer.

In some embodiments, the depot formulations provide herein can contain two different PLGA polymers mixed or blended in a % wt ratio of 95:5 to 50:50 (e.g., 85:15 to 50:50, 80:20 to 60:40, 90:10, 85:15, 80:20, 75:25, 70:30, 65:35, 60:40, 55:45 or 50:50% wt). In some embodiments, the polymer with the higher inherent viscosity can have a higher % wt than the polymer with the lower inherent viscosity. In some embodiments, the polymer with the higher inherent viscosity can have an ester end-group. Depot formulations can contain further polymers, including other linear or star shaped PLGA polymers, or poly(D,L-lactide-co-glycolide) (PLG) or polylactic acid (PLA) polymers, provided that favorable PK properties are retained.

The polypeptide content of the depot formulation (the loading) can be in a range of 1% to 30% (e.g., 10% to 25%, more preferred 15% to 20%. The loading is defined as the weight ratio of polypeptide to the total mass of the PLGA formulation.

Depot compositions can be manufactured aseptically, or can be manufactured non-aseptically and terminally sterilized (e.g., using gamma irradiation). Terminal sterilization can result in a product with the highest sterility assurance possible.

Depot compositions also can contain one or more pharmaceutical excipients that can modulate the release behavior of the polypeptide. Such excipients can be present in the composition in an amount of about 0.1% to about 50%. Suitable excipients include, without limitation, polyvinyl alcohol, polyvinyl pyrrolidone, carboxymethyl cellulose sodium, dextrin, PEG, surfactants such as poloxamers (also known as poly(oxyethylene-block-oxypropylene), poly(oxyethylene)-sorbitan-fatty acid esters commercially available under the trade name TWEEN®, sorbitan fatty acid esters, lecithins, inorganic salts such as zinc carbonate, magnesium hydroxide, magnesium carbonate, protamine, and natural or synthetic polymers bearing amine-residues such as polylysine.

Depot compositions can contain a mixture or blend of different polymers in terms of compositions, molecular weight and/or polymer architectures. A polymer blend is defined herein as a solid solution or suspension of two different linear polymers in one implant or microparticle. A mixture of depots is defined herein as a mixture of two depot-like implants or microparticles or semisolid formulations of different composition with one or more PLGAs in each depot. Pharmaceutical depot compositions in which two PLGAs are present as a polymer blend can be particularly useful.

Pharmaceutical depot compositions can be in the form of implants, semisolids (gels), liquid solutions, microparticles, or suspensions that solidify in situ once they are injected. The following paragraphs are focused on polymer microparticles, although the descriptions also are applicable for implants, semisolids, and liquids.

Microparticles can have a diameter from a few submicrons to a few millimeters (e.g., from about 0.01 micron to about 2 mm, about 0.1 micron to about 500 microns, about 10 to about 200 microns, about 10 to about 130 microns, or about 10 to about 90 microns).

In some embodiments, microparticles can be mixed or coated with an anti-agglomerating agent. Suitable anti-agglomerating agents include, for example, mannitol, glucose, dextrose, sucrose, sodium chloride, and water soluble polymers such as polyvinyl alcohol, polyvinyl pyrrolidone and PEG.

Microparticles can be manufactured using processes known in the art, such as coacervation or phase separation, spray drying, or water-in-oil (W/O), water-in-oil-in-water (W/O/W), or solids-in-oil-in-water (S/O/W) emulsion/suspension methods followed by solvent extraction or solvent evaporation. Emulsion/suspension methods can be particularly useful, and can include the following steps:

(i) preparing an internal organic phase, comprising
(a) dissolving a polymer or polymers in a suitable organic solvent (e.g., ethyl acetate, acetone, THF, acetonitrile, or a halogenated hydrocarbon such as methylene chloride, chloroform, or hexafluoroisopropanol) or solvent mixture, and optionally dissolving/dispersing suitable additives;
(b) dissolving/suspending/emulsifying a polypeptide in the polymer solution obtained in step (a);

(ii) preparing an external aqueous phase containing one or more stabilizers (e.g., poly(vinylalcohol), hydroxyethyl cellulose, hydroxypropyl cellulose, poly(vinyl pyrolidone), or gelatin) and optionally a buffer salt;

(iii) mixing the internal organic phase with the external aqueous phase to form an emulsion; and

(iv) hardening the microparticles by solvent evaporation or solvent extraction, washing the microparticles (e.g., with water), collecting and drying the microparticles (e.g., by freeze-drying or drying under vacuum), and sieving the microparticles (e.g., through 140 μm).

A dry microparticle composition can be terminally sterilized by gamma irradiation, either in bulk or after dispensing into the final container. In some embodiments, bulk sterilized microparticles can be resuspended in a suitable vehicle and dispensed into a suitable device such as double chamber syringe with subsequent freeze drying.

In some embodiments, microparticle depot compositions can include a vehicle to facilitate reconstitution. In addition, prior to administration, microparticles can be suspended in a suitable vehicle for injection (e.g., a water-based vehicle containing one or more pharmaceutical excipients such as mannitol, sodium chloride, glucose, dextrose, sucrose, or glycerin, and/or one or more non-ionic surfactants such as a poloxamer, poly(oxyethylene)-sorbitan-fatty acid ester, carboxymethyl cellulose sodium, sorbitol, poly(vinylpyrrolidone), or aluminium monostearate).

Also provided herein are articles of manufacture containing an M-ANP polypeptide or a pharmaceutical composition as described herein (e.g., a depot formulation containing an M-ANP polypeptide) in a vial, syringe, or other vessel. The article of manufacture also can include a transfer set and/or a water-based vehicle in a separate vessel, or the polypeptide/composition and vehicle can be separated in a double chamber syringe.

### Methods

This disclosure also provides methods for treating cardiovascular disorders (e.g., hypertension, resistant hypertension, and myocardial infarction) and metabolic disorders (e.g., type II diabetes and obesity) in a mammal by subcutaneous administration of a natriuretic polypeptide such as M-ANP. Subcutaneous methods for administering M-ANP to a mammal include, without limitation, DEPOT polymer delivery, drug patch delivery, injection, pump delivery, and micro/nanoparticle delivery. The studies described herein provide evidence that M-ANP can be particularly useful in such chronic delivery strategies. For example, M-ANP is resistant to in vitro proteolytic degradation, and in vivo (e.g., when given intravenously) it has greater blood pressure lowering, renal enhancing, and aldosterone suppressing properties than ANP. As described herein subcutaneously administered M-ANP is rapidly absorbed and promptly activates particulate guanylyl cyclase-A (GC-A), with resultant cGMP activation. Further, as discussed below, the area under the curve for cGMP immunoreactivity was greater for M-ANP compared to ANP, and the half-life of M-ANP (47 minutes) was significantly longer than the half-life of ANP (14 minutes). Concomitant with greater plasma cGMP activation, there also were significant and sustained reductions in systemic blood pressure following M-ANP administration, which were not observed with ANP. Thus, this disclosure provides methods for chronic, subcutaneous delivery of M-ANP to treat cardiovascular and metabolic diseases.

The term “treat” or “treatment” as used herein refers to prescribing, administering, or providing a medication to beneficially affect or alleviate one or more symptoms associated with a disease or disorder, or one or more underlying causes of a disease or disorder.

Before administering a polypeptide or composition provided herein to a mammal, the mammal can be assessed to determine whether or not the mammal has a need for treatment of a cardiovascular or metabolic disorder. After identifying a mammal as having a need for such treatment, the mammal can be treated with a composition provided herein. For example, a composition containing an M-ANP polypeptide can be administered to a mammal in any amount, at any frequency, and for any duration effective to achieve a desired outcome (e.g., to reduce one or more symptoms of a cardiovascular or metabolic disease, or to prevent or delay worsening of one or more such symptoms).

An effective amount of a composition can be any amount that reduces one or more symptoms of a cardiovascular or metabolic disorder in a mammal, without producing significant toxicity to the mammal. If a mammal fails to respond to a particular amount, then the amount can be increased by, for example, two fold, three fold, five fold, or ten fold. After receiving this higher concentration, the mammal can be monitored for both responsiveness to the treatment and toxicity symptoms, and adjustments made accordingly. The effective amount can remain constant or can be adjusted as a sliding scale or variable dose depending on the mammal's response to treatment.

As described herein, in some embodiments a composition containing a natriuretic polypeptide also can include insulin and/or an aldosterone inhibitor (e.g., spironolactone or eplerenone), such that the polypeptide and the insulin and/or aldosterone inhibitor can be administered simultaneously. In some embodiments, a method can include administering a polypeptide separately from insulin and/or an aldosterone inhibitor. In such embodiments, the insulin and/or aldosterone inhibitor can be administered once per day, more than once per day, or less than once per day. In some cases, the insulin or aldosterone inhibitor can be administered prior to administration of the natriuretic polypeptide (e.g., to stabilize the patient), while in other cases the insulin aldosterone inhibitor can be administered after the natriuretic polypeptide.

In general, dosing is dependent on the severity and responsiveness of the disease state to be treated. Those of ordinary skill in the art routinely determine optimum dosages and repetition rates. Optimum dosages can vary depending on the relative potency of the polypeptide, and generally can be estimated based on EC₅₀ found to be effective in in vitro and in vivo animal models. Dosages for an M-ANP polypeptide and, where included, insulin or an aldosterone inhibitor, can be from 0.001 μg to 10 g per kg of body weight (e.g., 0.001 μg/kg, 0.0025 μg/kg, 0.005 μg/kg, 0.01 μg/kg, 0.025 μg/kg, 0.05 μg/kg, 0.1 μg/kg, 2.5 μg/kg, 5 μg/kg, 10 μg/kg 25 μg/kg, 50 μg/kg, 100 μg/kg, 250 μg/kg, 500 μg/kg, 1 mg/kg, 2.5 mg/kg, 5 mg/kg, 10 mg/kg, 25 mg/kg, 50 mg/kg, 100 mg/kg, 250, or 500 mg/kg body weight). Following successful treatment, it may be desirable to have the patient undergo maintenance therapy to prevent the recurrence of the disease state.

A polypeptide can be administered once (e.g., by implantation or injection of a depot composition), or more than once (e.g., by repeated injections, or by use of a series of transdermal drug patches). When administered more than once, the frequency of administration can range from about four times a day to about once every other month (e.g., twice a day, once a day, three to five times a week, about once a week, about twice a month, about once a month, or about once every other month). In addition, the frequency of administration can remain constant or can be variable during the duration of treatment. Various factors can influence the actual frequency of administration used for a particular application. For example, the effective amount, duration of treatment, route of administration, and severity of condition may require an increase or decrease in administration frequency.

After administering a polypeptide or composition as provided herein to a mammal, the mammal can be monitored to determine whether or not the cardiovascular or metabolic disorder has improved. For example, a mammal can be assessed after treatment to determine whether or not one or more symptoms of the disorder have decreased. Any method can be used to assess improvements in function.

The methods provided herein can further include monitoring the concentration of the polypeptide in serum or plasma drawn from the patient. Blood can be drawn at regular intervals (e.g., every 15 minutes, 30 minutes, 1 hour, 2 hours, 4 hours, 6 hours, 10 hours, 12 hours, 20 hours, 22 hours, daily, biweekly, weekly, or monthly). Alternatively, blood can be drawn at random intervals. In still another aspect, an additional step may include creating a feedback loop by increasing or decreasing the amount of polypeptide administered after measuring its concentration.

Any suitable method can be used to measure serum levels of a polypeptide provided herein including, without limitation, mass spectrometry and immunological methods such as ELISA. An antibody used in an immunological assay can be, without limitation, a polyclonal, monoclonal, human, humanized, chimeric, or single-chain antibody, or an antibody fragment having binding activity, such as a Fab fragment, F(ab′) fragment, Fd fragment, fragment produced by a Fab expression library, fragment comprising a VL or VH domain, or epitope binding fragment of any of the above. An antibody can be of any type, (e.g., IgG, IgM, IgD, IgA or IgY), class (e.g., IgG1, IgG4, or IgA2), or subclass. In addition, an antibody can be from any animal including birds and mammals. For example, an antibody can be a human, rabbit, sheep, or goat antibody. Such an antibody can be capable of binding specifically to a polypeptide provided herein.

Antibodies can be generated and purified using any suitable methods known in the art. For example, monoclonal antibodies can be prepared using hybridoma, recombinant, or phage display technology, or a combination of such techniques. In some cases, antibody fragments can be produced synthetically or recombinantly from a gene encoding the partial antibody sequence. In some cases, an antibody fragment can be enzymatically or chemically produced by fragmentation of an intact antibody. An antibody directed against a polypeptide provided herein can bind the polypeptide at an affinity of at least 10⁴ mol⁻¹ (e.g., at least 10⁵, 10⁶, 10⁷, 10⁸, 10⁹, 10¹⁰, 10¹¹, or 10¹² mol⁻¹).

The invention will be further described in the following examples, which do not limit the scope of the invention described in the claims.

### EXAMPLES

### Example 1

### Materials and Methods

Natriuretic Peptide Synthesis and Reagents:

M-ANP and ANP were synthesized by Phoenix Laboratories (Burlingame, Calif.). Structures were confirmed by mass spectrometry, and high-performance liquid chromatography analysis confirmed purity to be >95%. Human GC-A and GC-B cDNA clones were purchased from Origene (Rockville, Md.).

Cells:

Human embryonic kidney 293 (HEK293) cells were stably transfected with either GC-A or GC-B using Lipofectamine (Invitrogen, Grand Island, N.Y.). Transfected cells were maintained in Dulbecco's modified Eagle's medium supplemented with 10% fetal bovine serum, 100 U/ml penicillin, 100 U/ml streptomycin, and 250 ug/ml G418 (all reagents from Invitrogen, Grand Island, N.Y.).

Cell Stimulation Studies and cGMP Assay:

Cells were plated in 6-well plates and treated as previously described (Tsuruda et al. (2002) Circ. Res., 91:1127-1134). Briefly, cells were incubated in Hank's balanced salt solution (Invitrogen, Carlsbad, Calif.) containing 20 mmol/L N-[2-hydroxyethyl]piperazine-N′[2-ethanesulfonic acid], 0.1% bovine serum albumin, and 0.5 mmol/L 3-isobutyl-1-methylzanthine (Sigma, St. Louis, Mo.). Treated cells received 10⁻⁸M or 10⁻¹⁰ M peptide for 10 minutes. Cells were lysed in 300 μl 6% TCA and sonicated for 10 minutes. The samples were extracted four times in 4 volumes of ether, dried, and reconstituted in 300 μl cGMP assay buffer. The samples were assayed using a competitive RIA cGMP kit (Perkin-Elmer, Boston, Mass.) as previously described (Steiner et al. (1970) Adv. Biochem. Psychopharmacol., 3:89-111).

In Vivo Study Protocol:

Studies were performed in accordance with the Animal Welfare Act and with approval of the Mayo Clinic Institutional Animal Care and Use Committee. A randomized crossover study was designed in which equimolar (2.225 nmol/kg) SQ M-ANP and SQ ANP were administered to conscious healthy adult canines (n=5) at 2-week intervals. Vehicle (SQ 0.9% normal saline) was administered to 3 canines serving as a control group. At least 7 days prior to the above studies, an arterial port was placed in the femoral artery as previously described (Cataliotti et al. (2008) Circulation, 118:1729-1736). After recovery from arterial port placement, canines were fed a fixed sodium diet of 100 mEq/d with ad libitum access to water for 5 days before initiation of studies. During this time, canines were acclimatized to standing calm in a sling. On the day of the experiment, the dogs were fasted and placed in a sling in a quiet room. Thirty minutes before the beginning of the experiment, the femoral artery port was accessed for continuous BP and heart rate assessment (iWorx, Dover, N.H.). After 30 minutes of acclimatization, the study protocol was started. Subcutaneous M-ANP, ANP or vehicle was administered at time 0 minutes. At time −15, 0, 5, 10, 15, 30, 45, 60, 75, 90, 120, 150, 180, 210, 240, 270, and 300 minutes, mean arterial pressure (MAP) and heart rate samples were obtained over a period of 1 minute. At the same times, arterial blood samples were obtained for measurement of plasma ANP and cGMP immunoreactivity. The study was completed 300 minutes after the SQ administration of M-ANP, ANP, or vehicle, and the arterial port was disengaged. Canines were allowed to recuperate for two weeks between studies.

Hormone Analysis:

Blood samples were collected in chilled EDTA tubes, immediately placed on ice, and centrifuged at 4° C. Plasma was stored at −80° C. until analysis. After extraction, plasma ANP immunoreactivity (Phoenix Pharmaceuticals, Burlingame, Calif.) was assessed by radioimmunoassay as previously described (Burnett et al. (1986) Science, 231:1145-1147). The cross-reactivity for M-ANP with the above assay is 100% (McKie et al. (2010) Hypertension, 56:1152-1159). Plasma cGMP (PerkinElmer, Waltham, Mass.) was assessed by radioimmunoassay (Steiner et al., supra).

Pharmacokinetics:

Pharmacokinetic profile of M-ANP compared to ANP was determined using PK Solutions software (Summit PK, Montrose, Colo.). Specific parameters that were determined included plasma half-life, maximum concentration (Cmax) and area under the curve (AUC).

Statistical Analyses:

Descriptive statistics are reported as mean±SE. Comparisons within a group were made by 1-way ANOVA for repeated measures followed by the Bonferroni multiple comparison posttest analysis when the global test was significant. Two-way ANOVA was used to compare the main group effects of M-ANP, ANP, and vehicle, followed by Bonferroni posttests. Unpaired t-test was performed for comparison between groups. GraphPad Prism 5 (GraphPad Software, La Jolla, Calif.) was used for the above calculations, and statistical significance was accepted as P<0.05.

Palmitoylation Protocol:

M-ANP was synthesized by conventional solid phase synthesis methods using Na-9-fluorenyl-methoxycarbonyl (Fmoc) amino acids and O-benzotriazole-N,N,N′,N′-tetramethyl-uronium-hexafluorophosphate (HBTU) activation chemistry on a CEM Liberty Peptide Synthesizer. Following final deprotection of the N-terminal Fmoc group with 20% piperidine in N,N-dimethylormamide (DMF) (v/v) for 20 minutes, the peptide-resin was washed with 3×10 ml DMF and then 3×10 ml dichloromethane (DCM) washes.

Palmitic acid (1 mmol; 256.4 mg/mmol) was dissolved in 5 ml DCM and added to the peptide-resin. A solution of 4 ml of DMF containing N-hydroxybenzotriazole (HOBt) (1 mmol; 153.1 mg/mmol) and HBTU (1 mmol; 379 mg/mmol) was then added to the peptide-resin, followed by addition of 2 mmol diisopropylethylamine (DIEA) (0.35 ml). The palmitoylation reaction was then allowed to proceed overnight (18 hours) at room temperature with constant vessel shaking. Following palmitoylation, the peptide-resin was washed with 3×10 ml DMF and 3×10 ml DCM washes, and then cleaved from the resin support using standard laboratory cleavage protocols.

The same general protocol is used to link other fatty acids (e.g., capric acid, lauric acid, myristic acid, palmitic acid, or steric acid) to M-ANP. It is noted that the fatty acid molecule can be attached to the free amino terminus or to any lysine side chain (an epsilon amino group). Further, a lysine residue for this attachment can be placed at either the C-terminal or N-terminal end of the peptide.

### Example 2

### In Vitro GC-A and GC-B cGMP Activation by M-ANP

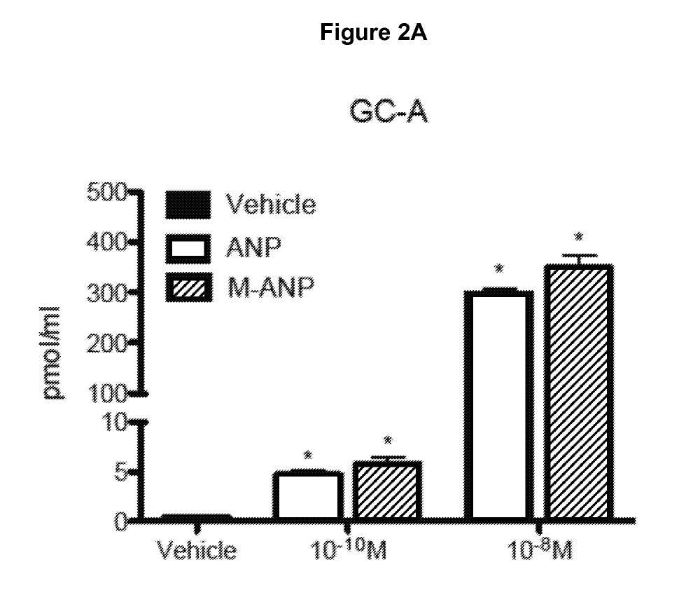

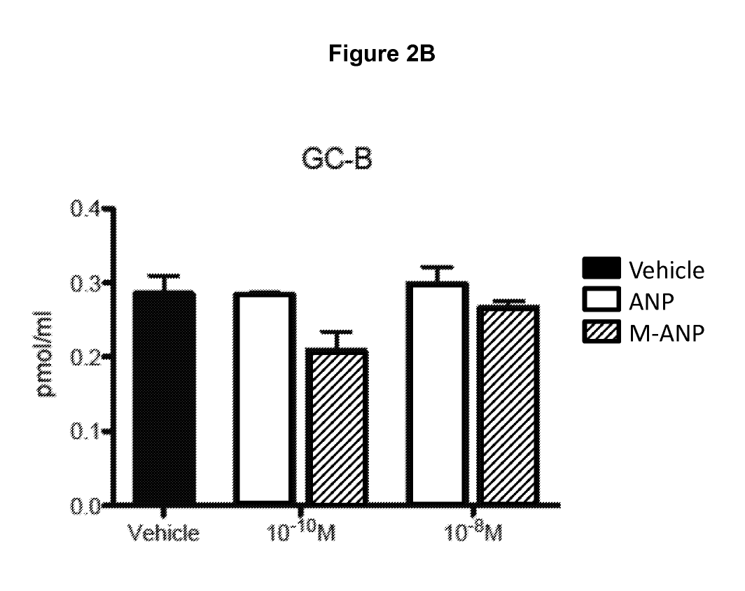

In vitro cGMP activation was assessed in HEK293 cells stably transfected with either GC-A or GC-B. Cells were treated with M-ANP or ANP (10⁻¹⁰ M and 10⁻⁸M) and compared to vehicle (no treatment). Results are shown in FIG. 2. Both M-ANP and ANP significantly activated cGMP via GC-A at 10⁻¹⁰ M and 10⁻⁸M as compared to vehicle. The degree of cGMP activation was similar between M-ANP and ANP at concentrations of 10⁻¹⁰ M (p=0.21) and 10⁻⁸M (p=0.86).

### Example 3

### ANP and cGMP Immunoreactivity In Vivo After ANP and M-ANP Treatment

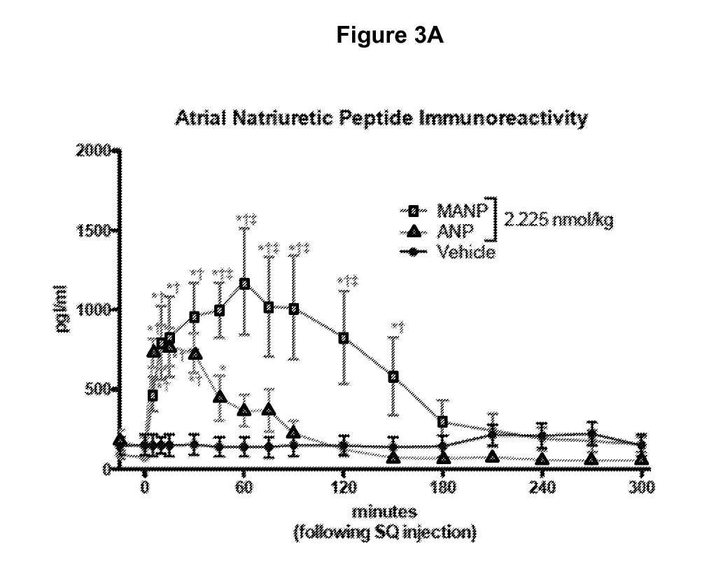

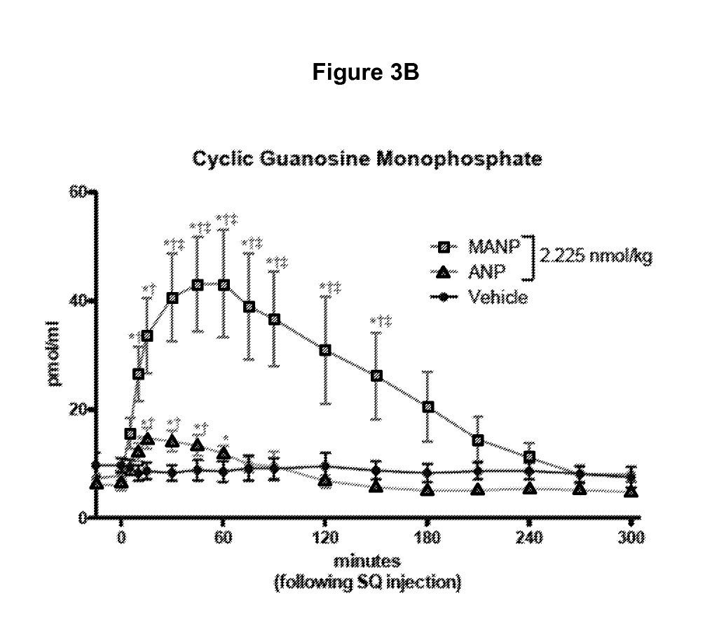

Plasma ANP immunoreactivity following SQ M-ANP and ANP administration is shown in FIG. 3A. Both M-ANP and ANP administration resulted in significant increases in plasma ANP immunoreactivity. Peak ANP immunoreactivity after M-ANP was 0.38±0.10 pmol/ml compared to ANP 0.25±0.04 pmol/ml (p>0.05). Peak ANP immunoreactivity occurred 60 minutes after administration of M-ANP, versus 15 minutes after ANP administration. Importantly, ANP immunoreactivity remained significantly elevated for 150 minutes following M-ANP treatment as compared to the 45 minute elevation of immunoreactivity following ANP administration. Additional pharmacokinetic data are reported in Table 1. The sustained increase in ANP immunoreactivity following M-ANP compared to ANP administration translated into significantly higher AUC for M-ANP (44.9±12.3 pmol-min/ml) compared to ANP (10.8±2.4 pmol-min/ml) (p=0.02). In addition, the half-life of SQ-administered M-ANP (47 minutes) was significantly greater (p=0.03) than ANP (14 minutes). There was no significant change in plasma ANP immunoreactivity following vehicle administration.

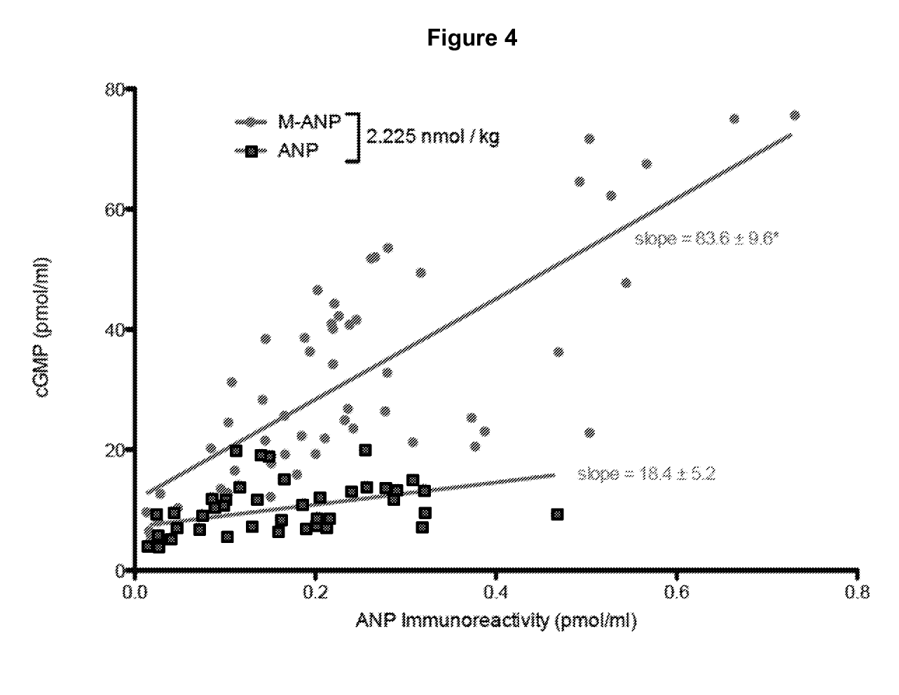

Consistent with increased plasma ANP immunoreactivity following SQ M-ANP and ANP administration, there were significant increases in plasma cGMP, the second messenger molecule of the natriuretic peptides (FIG. 3B). Maximum cGMP generation was greater for M-ANP (43.0±8.7 pmol/ml) than for ANP (14.7±1.9 pmol/ml) (p=0.01; Table 1). In addition, cGMP activation was sustained longer following M-ANP administration as compared to ANP administration (150 minutes vs. 60 minutes). Correspondingly, the AUC for cGMP was significantly greater following M-ANP (5,095±1,365 pmol-min/ml) compared to ANP (676±211 pmol-min/ml) (p<0.01; Table 1). There was no significant change in plasma cGMP in the vehicle group. FIG. 4 illustrates plasma cGMP generation for a given level of ANP immunoreactivity following M-ANP or ANP administration. Linear regression lines are shown and the slope for M-ANP is significantly greater than the slope for ANP (p<0.01).

### Example 4

### Mean Arterial Pressure After ANP and M-ANP Treatment

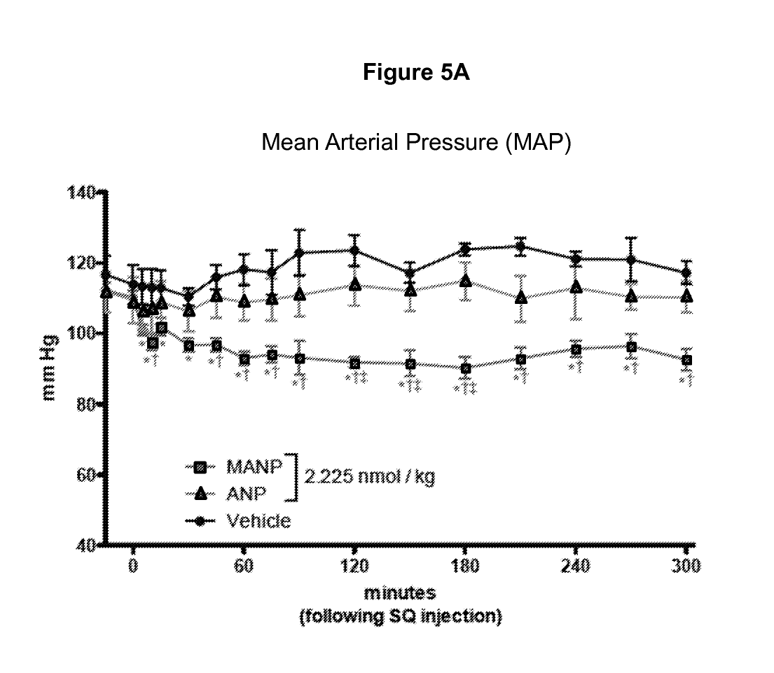

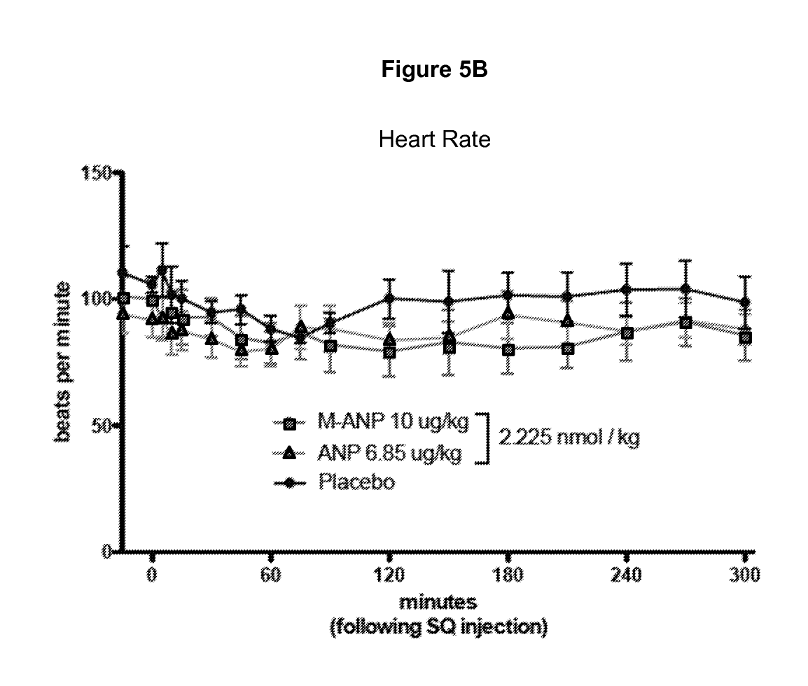

Mean arterial pressure (MAP) following SQ M-ANP and ANP administration is shown in FIG. 5. Baseline MAP was similar (p>0.05) among the three groups. Following SQ M-ANP there was a significant reduction in MAP within 5 minutes, which was sustained for the entirety of the experimental protocol (300 minutes). In contrast, there was no significant reduction in MAP following SQ ANP administration. Importantly, and despite a reduction in MAP following SQ M-ANP administration, heart rate was not significantly different when compared to ANP administration (FIG. 4B).

TABLE 1
Pharmacokinetics of subcutaneous administration of equimolar M-ANP and ANP

| | ANPi Cmax | ANPi AUC | Half-life | cGMP Cmax | cGMP AUC |
|---|---|---|---|---|---|
| | (pmol/ml) | (pmol-min/ml) | (min) | (pmol/ml) | (pmol-min/ml) |
| M-ANP | 0.38 ± 0.10 | 44.9 ± 12.3* | 47.4 ± 12.7* | 43.0 ± 8.7* | 5095 ± 1365* |
| ANP | 0.25 ± 0.04 | 10.8 ± 2.4 | 14.2 ± 1.5 | 14.7 ± 1.9 | 676 ± 211 |

ANPi, atrial natriuretic peptide immunoreactivity; AUC, area under the curve; cGMP, cyclic guanosine monophosphate; Cmax, maximum concentration.
*p < 0.05 vs. ANP as measured by unpaired t-test.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a cardiovascular or metabolic disorder in a mammal, comprising subcutaneously administering to the mammal a polypeptide comprising an amino acid sequence as set forth in SEQ ID NO:3, or an amino acid sequence as set forth in SEQ ID NO:3 having less than five amino acid additions, subtractions, and substitutions.

2. The method of claim 1, wherein the polypeptide comprises an amino acid sequence as set forth in SEQ ID NO:3 having less than five amino acid additions, subtractions, and substitutions.

3. The method of claim 1, wherein the polypeptide comprises an amino acid sequence as set forth in SEQ ID NO:3 having less than four amino acid additions, subtractions, and substitutions.

4. The method of claim 1, wherein the polypeptide comprises the amino acid sequence as set forth in SEQ ID NO:3.

5. The method of claim 1, wherein the polypeptide consists of the amino acid sequence set forth in SEQ ID NO:3.

6. The method of claim 1, wherein the cardiovascular disorder is selected from the group consisting of hypertension, resistant hypertension, and myocardial infarction.

7. The method of claim 1, wherein the metabolic disorder is selected from the group consisting of obesity and type II diabetes.

8. The method of claim 1, comprising administering the polypeptide via depot polymer, transdermal patch, injection, pump, microparticle, or nanoparticle.

9. The method of claim 1, further comprising administering to the mammal insulin and/or an aldosterone inhibitor.

10. The method of claim 9, wherein the aldosterone inhibitor is spironolactone or eplerenone.

11. The method of claim 9, comprising administering the polypeptide and the insulin and/or aldosterone inhibitor simultaneously.

12. The method of claim 9, comprising administering the polypeptide and the insulin and/or aldosterone inhibitor sequentially.

13. The method of claim 1, wherein the polypeptide is coupled to a fatty acid.

14. A method for treating a cardiovascular or metabolic disorder in a mammal, comprising subcutaneously administering to the mammal a composition comprising a polypeptide comprising an amino acid sequence as set forth in SEQ ID NO:3, or an amino acid sequence as set forth in SEQ ID NO:3 having less than five amino acid additions, subtractions, and substitutions.

15. The method of claim 14, wherein the polypeptide comprises an amino acid sequence as set forth in SEQ ID NO:3 having less than five amino acid additions, subtractions, and substitutions.

16. The method of claim 14, wherein the polypeptide comprises an amino acid sequence as set forth in SEQ ID NO:3 having less than four amino acid additions, subtractions, and substitutions.

17. The method of claim 14, wherein the polypeptide comprises the amino acid sequence as set forth in SEQ ID NO:3.

18. The method of claim 14, wherein the polypeptide consists of the amino acid sequence set forth in SEQ ID NO:3.

19. The method of claim 14, wherein the cardiovascular disorder is selected from the group consisting of hypertension, resistant hypertension, and myocardial infarction.

20. The method of claim 14, wherein the metabolic disorder is selected from the group consisting of obesity and type II diabetes.

21. The method of claim 14, comprising administering the composition via depot polymer, transdermal patch, injection, pump, microparticle, or nanoparticle.

22. The method of claim 14, further comprising administering to the mammal insulin and/or an aldosterone inhibitor.

23. The method of claim 22, wherein the aldosterone inhibitor is spironolactone or eplerenone.

24. The method of claim 22, wherein the composition comprises the polypeptide and the insulin and/or aldosterone inhibitor.

25. The method of claim 22, comprising administering the composition separately from the insulin and/or aldosterone inhibitor.

26. The method of claim 14, wherein the polypeptide is coupled to a fatty acid.

27. A method for making a conjugate comprising a polypeptide coupled to palmitic acid, comprising:
providing a polypeptide comprising an amino acid sequence as set forth in SEQ ID NO:3, or an amino acid sequence as set forth in SEQ ID NO:3 having less than five amino acid additions, subtractions, and substitutions, wherein said polypeptide is linked to a solid phase synthesis resin;
adding 1 mmol palmitic acid in 5 ml dichloromethane (DCM) to the peptide-resin;
adding 4 ml of dimethylformamide (DMF) containing 1 mmol N-hydroxybenzotriazole and 1 mmol O-benzotriazole-N,N,N′,N′-tetramethyl-uronium-hexafluorophosphate to the reaction mixture;
adding 2 mmol diisopropylethylamine to the reaction mixture;
incubating the reaction mixture for 18 hours at room temperature with shaking;
washing the peptide-resin with DMF;
washing the peptide-resin with DCM; and
cleaving the palmitoylated peptide from the resin.
